(19) United States
(12) Patent Application Publication
(10) Pub. No.: US 2013/0219998 A1
(43) Pub. Date: Aug. 29, 2013

(54) ROTATING SYSTEM AND METHOD FOR TESTING ENGINE LUBRICANTS

(71) Applicants: Abraham Robert DE KRAKER, Sugar Land, TX (US); Sharief Moghazy, Pearland, TX (US)

(72) Inventors: Abraham Robert DE KRAKER, Sugar Land, TX (US); Sharief Moghazy, Pearland, TX (US)

(73) Assignee: SHELL OIL COMPANY, Houston, TX (US)

(21) Appl. No.: 13/599,925

(22) Filed: Aug. 30, 2012

(51) Int. Cl. G01N 33/30

(52) U.S. Cl. CPC G01N 33/30
USPC 73/53.05

(57) ABSTRACT

Systems and methods for testing an engine lubricant are provided. The system includes a test assembly rotatably supportable on a platform. The test assembly includes a pair of spaced apart blocks and at least one test cylinder. The blocks include a support block and a test block. The support block has at least one cavity therein. The test block has a heat source applying heat to a test surface thereof. The test cylinder has the engine lubricant therein, and a closed end and an open end opposite thereto. The closed end is positionable in the cavity of the support block. The open end is positionable adjacent the test surface of the test block. The test cylinder is rotatable between a contact position and a non-contact position by the rotating frame such that the engine lubricant is selectively contactable with the test surface whereby deposits form on the test surface.

### BACKGROUND

The present disclosure relates generally to testing operations. More specifically, the present disclosure relates to techniques for testing fluids, such as lubricants used in engines.

Lubricants may be used in machinery to prevent friction between moving parts, such as pistons and cylinders of an engine. In some cases, deposits may form in the lubricants that may hinder the movement of the parts and, therefore, impact the performance of the engine. Lubricants may be configured to reduce the likelihood of deposits and/or affect the performance of the engine.

Lubricants may include a mix of oils and other additives. The composition of the lubricant may be selected to define properties which can be used to enhance performance of the machinery. For example, various engines may specify the use of a certain viscosity of lubricant under certain conditions, such as outdoor temperature. In another example, the composition of the lubricant (and/or its additives) may be selected to control the engine's tendency to oxidize and form deposits.

Designed experiments may be performed to compare lubricants having various compositions. The experiments may involve performing tests of various lubricants to determine how each lubricant will perform in an engine. The experiments may be performed using apparatuses that simulate the engine and provide controlled conditions for testing. Examples of tests are provided in U.S. Pat. Nos. 5,313,824, 5,287,731, 7,597,016, 6,571,611, 6,566,142 and 5,492,005.

In some cases, experiments may be conducted to determine properties of different lubricants which may affect the performance of the machinery. For example, tests may be performed to determine oxidation of lubricants. Examples of oxidation tests include TFOUT (Thin-Film Oxygen Uptake Test), PDSC (Pressurized Differential Scanning Calorimetry), Ciba Viscosity Increase Test (CVIT), HOOT (Hot Oil Oxidation Test), FOAT (Ford Oil Aging Test), and Oxidator (Oronite Oxidation) test. Tests may also be performed to detect deposit formation. Examples of deposit tests include inclined plane, panel coker, hot tube, sliding ring, and micro-oxidation. Facilities used in performing the various tests may be configured to simulate environments in which the lubricants are used.

### SUMMARY

In at least one aspect, the disclosure relates to a system for testing an engine lubricant. The system includes a test assembly rotatably supportable on a platform. The test assembly includes a pair of spaced apart blocks including a support block and a test block. The support block has at least one cavity therein. The test block has a heat source applying heat to a test surface thereof. The test cylinder has the engine lubricant therein, and has a closed end and an open end opposite thereto. The closed end is positionable in the cavity of the support block. The open end is positionable adjacent the test surface of the test block. The test assembly is rotatable between a contact position and a non-contact position by the rotating frame such that the engine lubricant is selectively contactable with the test surface whereby deposits form on the test surface.

The platform includes a stationary frame and a rotating frame. The rotating frame is rotatably supportable by the stationary frame. The support block is supported on the rotating frame. The test block has a higher temperature than the support block. The test assembly also includes a test plate operatively connectable to the test block. The test plate has the test surface thereon. The system also includes a motor, drive shaft and bearing selectively rotating the rotating frame about the stationary frame. The system may also include a controller selectively activating the motor to rotate the test assembly and/or selectively controlling the temperature of the heat sources. The test cylinder may be a test tube. The test cylinder may be sealed against the test surface using an o-ring or gasket. The heat source may be an electrical coil. The test assembly may also include a pair of cover plates positionable about the support block and the test block.

In another aspect, the disclosure relates to a system for testing an engine lubricant. The system includes a platform comprising a stationary frame and a rotating frame. The rotating frame is rotatably supportable by the stationary frame. The system also includes a test assembly rotatably supportable on the rotating frame. The test assembly includes a pair of spaced apart blocks comprising a support block and a test block. The support block has at least one cavity therein. The test block has a heat source applying heat to a test surface thereof. The test cylinder has the engine lubricant therein, and a closed end with an open end opposite thereto. The closed end is positionable in the cavity of the support block. The open end is positionable adjacent the test surface of the test block. The test cylinder is rotatable between a contact position and a non-contact position by the rotating frame such that the engine lubricant is selectively contactable with the test surface whereby deposits form on the test surface.

Finally, in another aspect, the disclosure relates to a method for testing an engine lubricant. The method involves providing a system for testing the engine lubricant. The system includes a test assembly rotatably supportable on a platform. The test assembly includes a pair of spaced apart blocks including a support block and a test block. The support block has at least one cavity therein. The test cylinder has the engine lubricant therein, and a closed end and an open end opposite thereto. The closed end is positionable in the cavity of the support block. The open end is positionable adjacent the test surface of the test block. The method also involves selectively applying heat to the pair of blocks, selectively rotating the test cylinder between a contact position and a non-contact position by the rotating frame such that the engine lubricant is selectively contactable with the test surface whereby deposits form on the test surface, and examining the deposits formed on the test surface.

The selectively rotating may be performed at selected intervals. The selectively applying may involve selectively applying heat to the test block and/or the support block. The method may also involve analyzing the deposits formed on the test surface.

### BRIEF DESCRIPTION OF THE DRAWINGS

So that the above recited features and advantages of the disclosure may be understood in detail, a more particular description of the disclosure, briefly summarized above, may be had by reference to the embodiments thereof that are illustrated in the appended drawings. It is to be noted, however, that the appended drawings illustrate only typical embodiments of this disclosure and are, therefore, not to be considered limiting of its scope. The figures are not necessarily to scale, and certain features and certain views of the figures may be shown exaggerated in scale or in schematic in the interest of clarity and conciseness.

FIG. 1 is a schematic diagram depicting a system with a rotating test assembly for testing engine lubricants in accordance with the present disclosure;

FIG. 2 is a schematic, exploded view of the test assembly of FIG. 1 in accordance with the present disclosure;

FIGS. 3A-3C are schematic diagrams depicting the test assembly of Figure 1 in a non-contact, a partial contact and a full contact position, respectively, in accordance with the present disclosure;

FIG. 4 is a detailed view of a test plate of the system of FIG. 1 in accordance with the present disclosure; and

FIGS. 5 is a flow chart depicting a method for testing engine lubricants in accordance with the present disclosure.

### DETAILED DESCRIPTION

The description that follows includes exemplary apparatuses, methods, techniques, and instruction sequences that embody techniques of the inventive subject matter. However, it is understood that the described embodiments may be practiced without these specific details.

The disclosure relates to techniques for testing lubricants, such as those used in engines. These techniques may involve the use of a test assembly rotatable such that testing lubricant therein is selectively positionable between a non-contact and a contact position relative to a heated test surface of the test assembly. The test surface may be examined for the formation of deposits. The experiments may be performed over time and selectively controlled to define a test cycle. The system is configured to incorporate characteristics of a combustion engine, such as an oil sump at adjustable temperatures with a hot surface to simulate a piston at a secondary, higher temperature. The test simulates an engine by moving oil from a lower temperature sump to a higher temperature engine piston, thereby creating deposits.

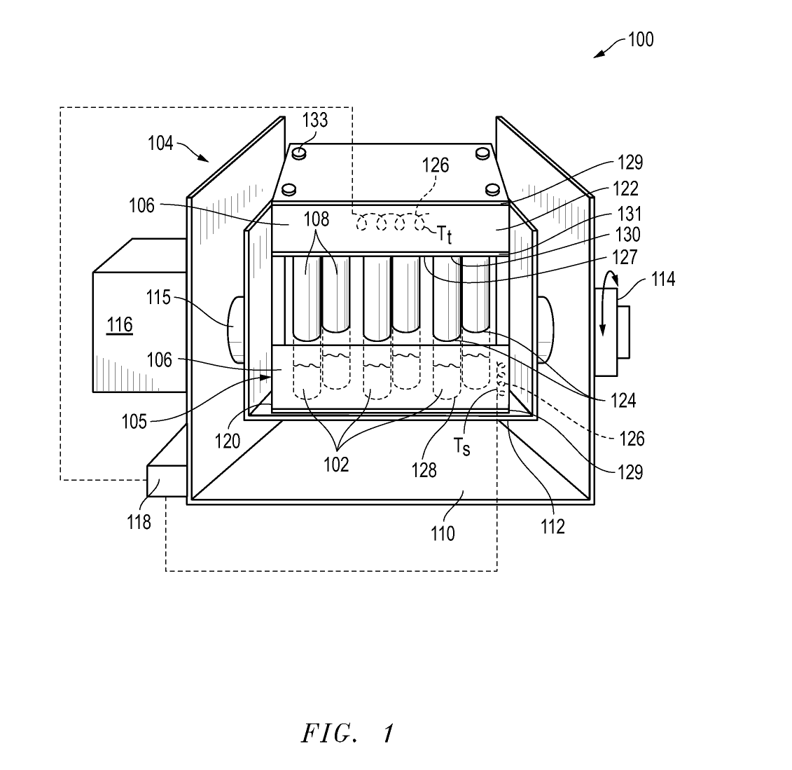

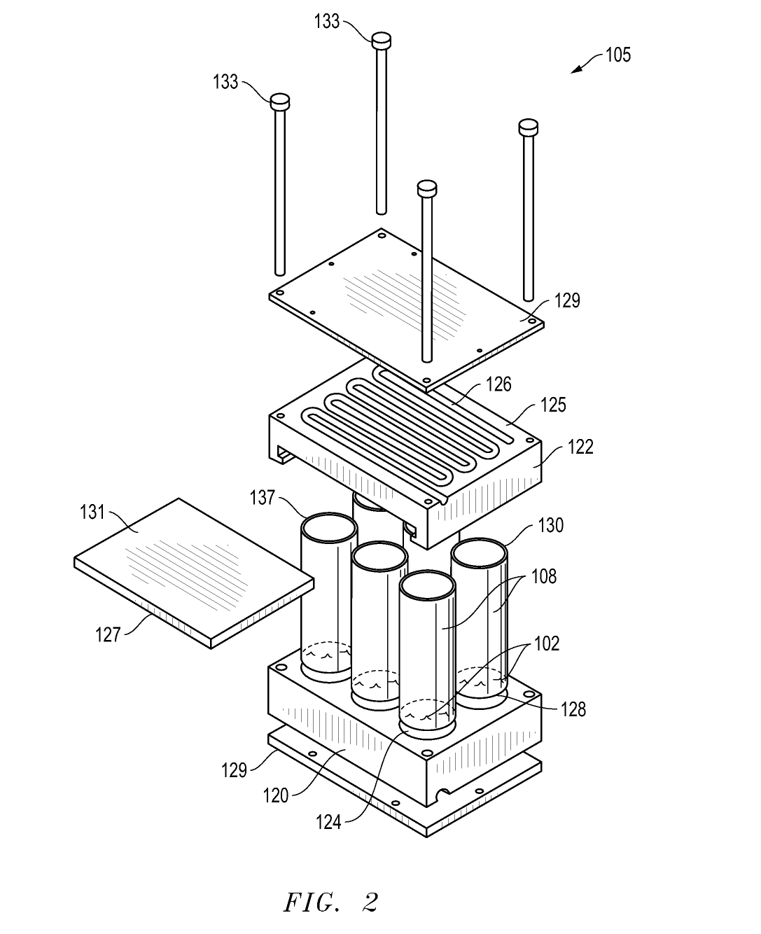

FIGS. 1 and 2 illustrate a system 100 usable for evaluating a lubricant 102. The system includes a platform 104 and a rotating test assembly 105. The platform 104 includes a stationary frame 110 and a rotating frame 112. The stationary frame 110 is fixedly positionable on a surface, such as a countertop. The rotating frame 112 is rotatably supportable by the stationary frame 110 as indicated by the curved, bi-directional arrows.

The system 100 also includes a bearing 114, a drive shaft 115, and a motor 116 operatively connectable to the platform 104. The bearing 114 rotatably supports the drive shaft 115. The drive shaft 115 is operatively connected to the rotating frame 112 for rotation relative to the stationary frame 110. The motor 116 is operatively connectable to the drive shaft 115 for selectively rotating the rotating frame 112 about the stationary frame 110.

The controller 118 is operatively connected to the motor 116 to selectively activate the motor 116 to rotate the rotating frame 112. The rotating frame 112 may be rotated as desired, for example, in a predetermined cycle for a given duration. In some cases, the rotating frame 112 may be rotated by the controller 118 to a desired angle for a desired duration.

The test assembly 105 includes a pair of spaced apart blocks 106 and test cylinders 108. The pair of spaced apart blocks 106 is supportable by the rotating frame 112. The pair of blocks 106 includes a support block 120 and a test block 122. The support block 120 is positionable about a bottom of the rotating frame 112. The test block 122 is positioned a distance above the support block 120 and is parallel thereto. A pair of cover plates 129 may be positioned about the pair of test blocks 106 to apply support thereto. As shown, a first cover plate 129 is positioned on an outer surface of the test block 122 and a second cover plate 129 is positioned on a frame surface of the support block 120. Bolts or other fasteners 133 may be provided to secure the plates 129 and the pair of blocks 106 together.

The pair of blocks 106 also has a heat source 126 applying heat thereto. The heat source 126 may be, for example, an electrical coil positioned in the pair of blocks 106. The blocks 106 may be provided with grooves 125 for receiving the coil 126. As shown, one heat source 126 is positioned in each of the pair of blocks 106, but may only be in the test block 122. The heat source 126 may apply heat to the pair of blocks 106. The test block 122 has a test surface 127 heatable by the heat source 126.

The heat source 126 may be operatively connected to the controller 118 for controlling heating thereof. The temperature of each of the pair of blocks 106 may be selectively controlled to provide a desired amount of heat during the test. In a given example, the test block 122 may have a higher temperature Tₜ than a temperature Tₛ of the support block 120. For example, the test block 122 may be heated to about 300 C and the support block 120 may be heated to about 160 C.

The support block 120 has cavities 124 therein. The test cylinders 108 are positionable in the cavities 124. The test cylinders 108 have the engine lubricant 102 therein. The test lubricant 102 may be any lubricant (e.g., motor oil) or mix of lubricants to be tested. A mixture of lubricants may include the test lubricant 102 and another lubricant, such as a taxi oil. A taxi oil refers to lubricants that have been previously used over a period of time, for example, in a taxi cab. The taxi oil may be added in desired ratios with the test lubricant to speed up the oxidation and/or the test process. Gas (e.g., air or NO2) may also be added to the fluid to facilitate oxidation and/or testing. Other lubricants, gases and/or additives may also be provided as desired to achieve the desired test conditions for evaluating the test lubricant 102.

The test cylinders 108 each have a closed end 128 and an open end 130 opposite thereto. The closed end 128 is positionable in the cavities 124 of the support block 120. The open end 130 is positionable adjacent the test block 122. A gasket 137 may be provided to seal the open end 130 adjacent the test block 122. The test block 122 may include a test plate 131 operatively connectable thereto. The test block 122 may have the test surface 127 thereon positionable adjacent the open end 130 of the test cylinder 108.

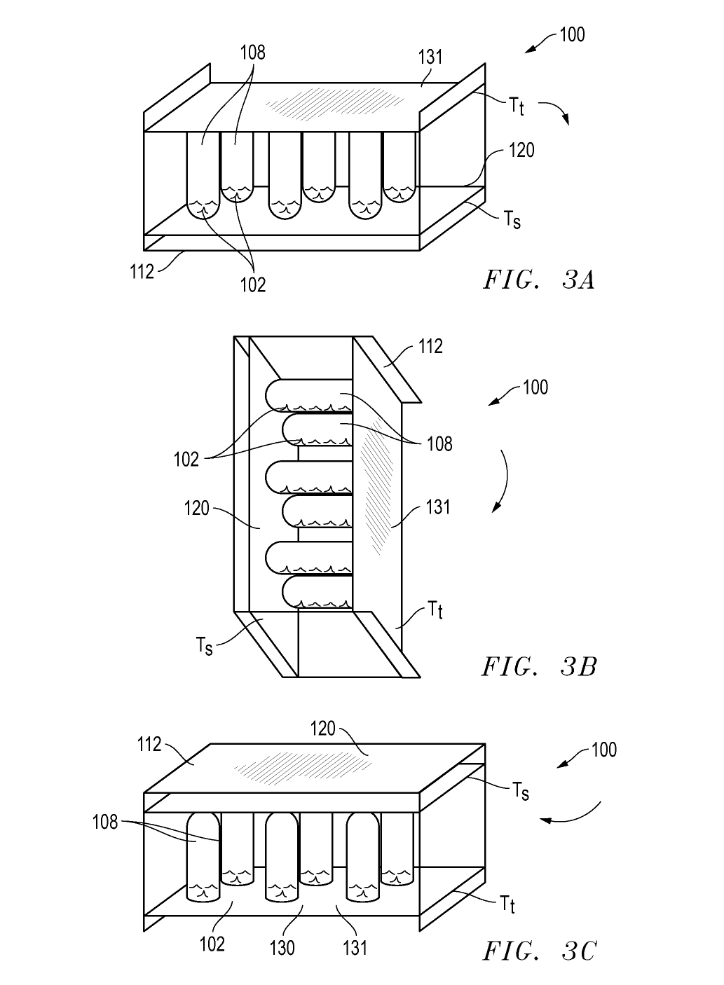

FIGS. 3A-3C depict the test assembly 105 during a test cycle. As shown in these figures, the test assembly 105 is rotatable by the rotating frame 112 such that the test lubricant 102 is movable therein. The test assembly 105 is rotated from an initial or non-contact position in FIG. 3A to an intermediate (or horizontal) or partial contact position in FIG. 3B and to an inverted or full contact position in FIG. 3C by the rotating frame 112. As demonstrated by these figures, the rotating frame 112 may be rotated between various positions such that the engine lubricant 102 is selectively contactable with the test surface 130.

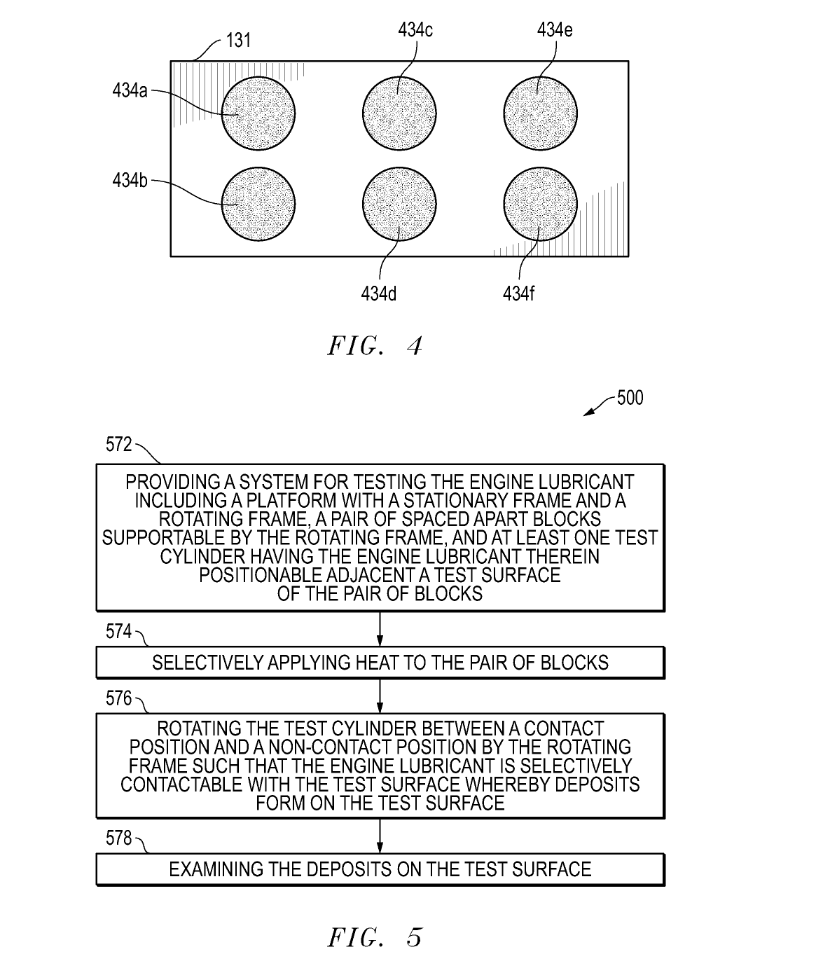

Over time, the engine lubricant 102 will form deposits on the test surface 130 as shown in FIG. 4. FIG. 4 depicts the test plate 131 in greater detail after a test is performed. As shown in this view, the test surface 130 of the test plate 131 has been exposed to engine lubricants 102 in test cylinders 106 (FIGS. 1-3C). The test lubricant 102 in each of the test cylinders 106 forms the deposits 434a-f. As shown in this figure, each test cylinder may have different lubricants or different test cycles resulting in different amounts of deposits 434a-f forming thereon. The various deposits 434a-f may be compared and analyzed.

FIG. 5 depicts a method 500 of testing an engine lubricant. The method involves 572—providing a system for testing the engine lubricant as provided herein. The system may include a platform and a test assembly rotatably connected thereto. The test assembly includes a pair of spaced apart blocks supportable by the rotating frame and at least one test cylinder having the engine lubricant therein positionable adjacent a test surface of the pair of blocks. The method also involves 574—selectively applying heat to the pair of blocks (the test block and the test surface having a higher temperature than the support block), 576—rotating the test cylinder between a contact position and a non-contact position by the rotating frame such that the engine lubricant is selectively contactable with the test surface whereby deposits form on the test surface, and 578—examining the deposits on the test surface.

The rotating may be performed at selected intervals. The method may also involve applying heat to the test block and/or the support block, and/or analyzing the deposits on the test surface. The method may be repeated as desired and performed in any order.

While the embodiments are described with reference to various implementations and exploitations, it will be understood that these embodiments are illustrative and that the scope of the inventive subject matter is not limited to these embodiments. Many variations, modifications, additions and/or improvements are possible. For example, one or more test cylinders with one or more lubricants may be selectively moved to the contact position in a desired test cycle.

Plural instances may be provided for components, operations or structures described herein as a single instance. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the inventive subject matter.

What is claimed is:

1. A system for testing an engine lubricant, comprising:
a test assembly rotatably supportable on a platform, the test assembly comprising:
a pair of spaced apart blocks comprising a support block and a test block, the support block having at least one cavity therein, the test block having a heat source applying heat to a test surface thereof; and
at least one test cylinder having the engine lubricant therein, the at least one test cylinder having a closed end and an open end opposite thereto, the closed end positionable in the at least one cavity of the support block, the open end positionable adjacent the test surface of the test block, the at least one test cylinder rotatable between a contact position and a non-contact position by a rotating frame such that the engine lubricant is selectively contactable with the test surface whereby deposits form on the test surface.

2. The system of claim 1, wherein the platform comprises a stationary frame and a rotating frame, the rotating frame rotatably supportable by the stationary frame, the support block supportable on the rotating frame.

3. The system of claim 1, wherein the support block has a heat source applying heat thereto, the test block having a higher temperature than the support block.

4. The system of claim 1, wherein the test assembly further comprises a test plate operatively connectable to the test block, the test plate having the test surface thereon.

5. The system of claim 1, further comprising a motor, drive shaft and bearing selectively rotating the rotating frame about the stationary frame.

6. The system of claim 5, further comprising a controller selectively activating the motor to rotate the test assembly.

7. The system of claim 1, further comprising a controller selectively controlling a temperature of the test surface.

8. The system of claim 1, wherein the at least one test cylinder comprises at least one test tube.

9. The system of claim 1, wherein the heat source comprises an electrical coil.

10. The system of claim 1, wherein the test assembly further comprises a pair of cover plates positionable about the support block and the test block.

11. A system for testing an engine lubricant, comprising:
a platform comprising a stationary frame and a rotating frame, the rotating frame rotatably supportable by the stationary frame; and
a test assembly rotatably supportable on the rotating frame, the test assembly comprising:
a pair of spaced apart blocks comprising a support block and a test block, the support block having at least one cavity therein, the test block having a heat source applying heat to a test surface thereof; and
at least one test cylinder having the engine lubricant therein, the at least one test cylinder having a closed end and an open end opposite thereto, the closed end positionable in the at least one cavity of the support block, the open end positionable adjacent the test surface of the test block, the at least one test cylinder rotatable between a contact position and a non-contact position by a rotating frame such that the engine lubricant is selectively contactable with the test surface whereby deposits form on the test surface.

12. A method for testing an engine lubricant, comprising:
providing a system for testing the engine lubricant comprising:
a test assembly rotatably supportable on a platform, the test assembly comprising:
a pair of spaced apart blocks comprising a support block and a test block, the support block having at least one cavity therein; and
at least one test cylinder having the engine lubricant therein, the at least one test cylinder having a closed end and an open end opposite thereto, the closed end positionable in the at least one cavity of the support block, the open end positionable adjacent a test surface of the test block;
selectively applying heat to the pair of blocks;
selectively rotating the at least one test cylinder between a contact position and a non-contact position by a rotating frame such that the engine lubricant is selectively contactable with the test surface whereby deposits form on the test surface; and
examining the deposits formed on the test surface.

13. The method of claim 12, wherein the selectively rotating is performed at selected intervals.

14. The method of claim 12, wherein the selectively applying comprises selectively applying heat to one of the test block, the support block and combinations thereof.

15. The method of claim 12, further comprising analyzing the deposits formed on the test surface.
